(19) United States
(12) Patent Application Publication
(10) Pub. No.: US 2016/0228008 A1
(43) Pub. Date: Aug. 11, 2016

(54) IMAGE DIAGNOSIS DEVICE FOR PHOTOGRAPHING BREAST BY USING MATCHING OF TACTILE IMAGE AND NEAR-INFRARED IMAGE AND METHOD FOR AQUIRING BREAST TISSUE IMAGE

(71) Applicant: DG TECH HOLDINGS CO.,LTD, Gyeongsangbuk-do (KR)

(72) Inventor: Jong-Ha LEE, Daegu (KR)

(21) Appl. No.: 15/022,574

(22) PCT Filed: Sep. 17, 2014
(86) PCT No.: PCT/KR2014/008632

§ 371 Date: Mar. 17, 2016

(30) Foreign Application Priority Data
Sep. 17, 2013 (KR) 10-2013-0112212

(51) Int. Cl. A61B 5/00; A61B 5/145; A61B 5/1455

(52) U.S. Cl. CPC A61B 5/0091; A61B 5/7282; A61B 5/7425; A61B 5/1455; A61B 5/14546; A61B 2562/0247; A61B 2562/0233; A61B 2503/40

(57) ABSTRACT

According to an image diagnosis device for photographing a breast by using the matching of a tactile image and a near-infrared image and a method for acquiring a breast tissue image, provided in the present invention, a tactile image and a near-infrared image are simultaneously acquired through a tactile image acquisition unit and a near-infrared image acquisition unit, the acquired images are mapped to a pre-stored breast model so as to match the two images, and a simple, economic and accurate early diagnosis for a breast cancer is enabled without any help from a doctor by simultaneously deriving an elasticity distribution and a hemoglobin distribution of the breast.

### TECHNICAL FIELD

The present invention relates to an image diagnosis device for photographing breasts and a method for acquiring a breast tissue image an more particularly, to an image diagnosis device for photographing breasts by using matching of a tactile image and a near-infrared image and a method for acquiring a breast tissue image.

### BACKGROUND ART

Breast cancer is a disease frequently occurring in middle-aged women and early diagnoses and treatment can largely reduce the lethality of the disease. However, in most cases there is no specific symptom in the early stage of breast cancer, aside from a painless lump that may be found by palpation. The accuracy of such self-diagnosis depends on the individual's skill and sensitivity. Accordingly, doctors recommended people to have regular examinations for breast cancer.

As devices for diagnosing breast cancer through images, there are an X-ray mammography system, an ultrasonic scanner, and a magnetic resonance imaging system. Among these devices, the X-ray mammography system is the most widely used for early diagnosis of breast cancer (see Korean Patent Application Nos. 10-2009-0096934 and 10-2008-0004564. The X-ray mammography system, which uses difference in transmission coefficient of X-rays according to tissues, effectively discriminates tissues that absorb X-rays well, for example, calcified tissues. Calcified tissues have a high possibility developing into cancer tissues, so early identification of calcified tissues largely contributes to preventing breast cancer.

However, the X-ray mammography system follows a way of visually recognizing images of breast cancer tissues that are lower in contrast than normal tissues, so diagnoses is required by specialized and skilled doctors. Accordingly, there is a problem that the accuracy of diagnosis may depend on the skillfulness of specialized doctors and the examination cost is high. Further, since electromagnetic waves in the range of wavelengths shorter than that of ultraviolet rays are used and images are made on two-dimensional projection plates, there is a limit in showing images and there is a danger of exposure to radiation. As the rate of breast cancer has been increasing due to changes in westernized eating habits, there is a need for a digitalized self-diagnostic system without the defects of the X-ray mammography system.

On the other hand, it is possible to determine that the disease is closely connected with a change in elasticity of a tissue from fact that when touching a tissue changed from breast cancer it feels like a lump that cannot be easily deformed by pressure. In the related art, spatial resolution was increased by obtaining tactile data using a capacitive sensor and then matching images through an algorithm (Heever, D. I., Schreve, K., and Scheffer, C, (2009). “Tactile sensing using force sensing resistors and a super-tension algorithm”. IEEE Sensors Journal, 9(1):29-35.). However, this method has a problem that it is required to continuously acquire several images and there is a limit in acquisition of high resonance.

Therefore, the inventor(s) proposes new optical image diagnosis equipment that can diagnose a breast cancer with high accuracy and low examination and construction costs without a danger of exposure to radiation by using a way of simultaneously acquiring a near-infrared image and tactile data with high spatial resolution and then matching them.

### DISCLOSURE

### Technical Problem

The present invention has been made in an effort to solve the problems with the methods proposed before and an object of the present invention is to provide an image diagnosis device for a breast cancer by using matching of a tactile image and a near-infrared image, the device being able to achieve a simple, economic, and accurate early diagnosis for breast cancer without any help from a doctor by simultaneously acquiring a tactile image and a near-infrared image from a tactile image acquisition unit and a near-infrared image acquisition unit and matching the acquired images by mapping them to a pre-stored breast model to simultaneously derive an elasticity distribution and a hemoglobin distribution of the breast, and a method for acquiring a breast tissue image.

### Technical Solution

In order to achieve the above object, according to one aspect of the present invention, there is provided a sensing probe for scanning a breast, the probe including: a tactile image acquisition unit acquiring a tactile image by collecting elasticity distribution information of a breast through a tactile sensor; and a near-infrared image acquisition unit acquiring a near-infrared image by radiating near-infrared light to a breast.

The tactile sensor of the tactile image acquisition unit may be any one selected from Ct group including a pressure sensor, a piezoelectric sensor, and an optical tactile sensor.

The tactile image acquisition unit may collect elasticity distribution information of a squeezed breast when a compression paddle squeezes the breast with a predetermined compression rate, and the near-infrared image acquisition unit may collect hemoglobin distribution information of a breast using a property of hemoglobin absorbing near-infrared wavelengths.

According to another aspect of the present invention, there is provided an image diagnosis device for photographing breast by using matching of a tactile image and a near-infrared image, the device including: an image acquisition module including a tactile image acquisition unit acquiring a tactile image by collecting elasticity distribution information of a breast through a tactile sensor and a near-infrared image acquisition unit acquiring a near-infrared image by radiating near infrared light to a breast; an image processing module matching and displaying the images acquired by the tactile image acquisition unit and the near-infrared image acquisition unit on same coordinates; and an image display module displaying the images processed by the image processing module.

The tactile sensor of the tactile image acquisition unit may be any one selected from a group including a pressure sensor, a piezoelectric sensor, and an optical tactile sensor.

The tactile image acquisition unit may collect elasticity distribution information of a squeezed breast when a compression paddle squeezes the breast with a predetermined compression rate, and the near-infrared image acquisition unit may collect hemoglobin distribution information, of a breast using a property of hemoglobin absorbing near-infrared wavelengths.

The image processing module may derive a color map in accordance with intensity of elasticity using the elasticity distribution information of a breast collected by the tactile image acquisition unit and derive a hemoglobin map using the hemoglobin distribution information of a breast collected by the near-infrared image acquisition unit.

The image processing module may match two images by mapping the tactile image and the near-infrared image to pre-stored breast model.

The pre-stored breast model may be derived from an electronic medical record of a corresponding patient.

The image processing module may make the elasticity distribution information of a breast acquired by the tactile image acquisition unit into a breast tissue elasticity map by applying a finite elements method, an inversion algorithm, and a forward algorithm to the elasticity distribution information, using an artificial neural network.

The image display module may include any one selected from a group of a CRT, an LCD, an LED, an OLED, and a PDP.

The image diagnosis device of claim may further include: a computer aided diagnosis module including: a tumor detection unit detecting a portion where elasticity is a predetermined level or less or hemoglobin is a predetermined level or more from an image acquired by the image acquisition unit; a feature extraction unit extracts discriminated features by comparing the portion detected by the tumor detection unit with a normal tissue; and a search & classifying unit searching and classifying features extracted by the feature extraction unit through a search engine.

According to another aspect of the present invention, there is provided a method of for acquiring a breast tissue image by using matching of a tactile image and a near-infrared image, the method including: (1) a step of squeezing a breast with a compression paddle; (2) a step of acquiring a tactile image by collecting elasticity distribution information of the squeezed breast through a tactile sensor and acquiring a near-infrared image including hemoglobin distribution information of the breast; (3) a step of matching and displaying the tactile image and the near-infrared image acquired in the step (2) on same coordinates; and (4) a step of displaying the images processed in the step (3) on an image display module.

The tactile sensor in the step (2) may be any one selected from a group including a pressure sensor, a piezoelectric sensor, and an optical tactile sensor.

The step (2) may include a step of deriving a color map in accordance with intensity of elasticity using the elasticity distribution information of a breast and deriving a hemoglobin map using the hemoglobin distribution information of a breast.

The step (2) may include a step of making the elasticity distribution information of a breast acquired by the tactile image acquisition unit into a breast tissue elasticity map by applying a finite elements method, an inversion algorithm, and a forward algorithm to the elasticity distribution information, using an artificial neural network.

The step (3) may be a step of matching two images by mapping the tactile image and the near-infrared image to pre-stored breast model.

The pre-stored breast model may be derived from an electronic medical record of a corresponding patient.

The method may further include: after the step (4), (5) detecting a portion where elasticity is a predetermined level or less or hemoglobin a predetermined level or more; (6) extracting discriminated features by comparing the portion detected in the step (5) with a normal tissue; and (7) searching and classifying the features extracted in the step (6) through a search engine

The method may be used for animals.

### Advantageous Effects

According to the image diagnosis device for photographing breast by using matching of a tactile image and a near-infrared image and the method of for acquiring a breast tissue image proposed by the present invention, it is possible to achieve a simple, economic, and accurate early diagnosis for a breast cancer without any help from a doctor by simultaneously acquiring a tactile image and a near-infrared image from a tactile image acquisition unit and a near-infrared image acquisition unit and matching the acquired images by mapping them to a pre-stored breast model to simultaneously derive an elasticity distribution and a hemoglobin distribution of the breast, and a method for acquiring a breast tissue image.

### DESCRIPTION OF DRAWINGS

FIG. 1 is a diagram showing the configuration of a sensing probe for scanning a breast according to an embodiment of the present invention.

FIG. 2 is a diagram showing the configuration of an image diagnosis device for photographing a breast by using matching of a tactile image and a near-infrared image according to an embodiment of the present invention.

FIG. 3 is a picture of a tactile image acquired by an image diagnosis device for photographing a breast by using matching of a tactile image and a near-infrared image according to an embodiment of the present invention, an elasticity map created by mapping the tactile image to a breast model.

FIG. 4 is a picture of a near-infrared image acquired by an image diagnosis device for photographing a breast by using matching of a tactile image and a near infrared image according to an embodiment of the present invention.

FIG. 5 is a picture of a hemoglobin map crated from a near-infrared image acquired by an image diagnosis device for photographing a breast by using matching of a tactile image and a near-infrared image according to an embodiment of the present invention.

FIG. 6 is a diagram showing the configuration of an image diagnosis device for photographing a breast by using matching of a

tactile image and a near-infrared image according to another embodiment of the present invention.

FIG. 7 is a diagram showing a process of image diagnosis for a breast by using matching of a tactile image and a near-infrared image according to another embodiment of the present invention.

FIG. 8 is a diagram showing a diagnosis system using an image diagnosis device for photographing a breast by using matching of a tactile image and a near-infrared image according to an embodiment of the present invention.

FIG. 9 is a flowchart showing a method for acquiring a breast tissue image by using matching of a tactile image and a near-infrared image according to an embodiment of the present invention.

FIG. 10 is a method for acquiring a breast tissue image by using matching of a tactile image and a near-infrared image according to another embodiment of the present invention.

### DESCRIPTION OF THE REFERENCE NUMERALS IN THE DRAWINGS

10: Tactile image acquisition unit
11: Tactile sensor
20: Near-infrared image acquisition unit
21: Lamp
100: Image acquisition module
200: Image processing module
300: Image display module
400: Computer aided diagnosis module
410: Tumor portion detection unit
420: Feature detection unit
430: Searching and classifying unit
S100: Step of squeezing a breast with a compression paddle
S200: Step of acquiring tactile image by collecting elasticity distribution information of squeezed breast with tactile sensor and acquiring near-infrared image including hemoglobin distribution information by radiating near-infrared light to squeezed breast
S300: Step of displaying tactile image and near-infrared image acquired in step S200 at same coordinates by matching the images
S400: Displaying image processed in step S300 on image display module
S500: Detecting portion where elasticity is predetermined level or less hemoglobin is predetermined level or more
S600: Extracting discriminated features by comparing portion detected in step S500 with normal tissue
S700: Searching and classifying features extracted in step S600 through search engine

### BEST MODE

Hereinafter, the preferred embodiment will be described with reference to the accompanying drawing's for those skilled in the art to be able to easily accomplish the present invention. However, in the following description of the preferred embodiment of the present invention, functions or configurations determined as unnecessarily making the present invention unclear are not described in detail. Further, the components having similar functions and actions are indicated by the same or similar reference numerals throughout the drawings.

Throughout the specification, it should be understood that when one element is referred to as being “connected to” another element, it may be “connected directly to” another element or “connected electrically to” another element, with the other element therebetween. Further, unless explicitly described otherwise, “comprising” any components will be understood to imply the inclusion of other components rather than the exclusion of any other components.

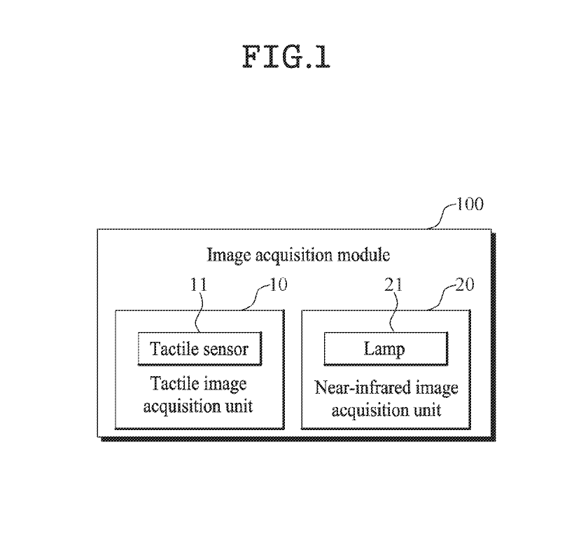

FIG. 1 is a diagram showing the configuration of a sensing probe for scanning a breast according to an embodiment of the present invention. As shown in FIG. 1, a sensing probe for scanning a breast according to an embodiment of the present invention may include a tactile image acquisition unit 10 and a near-infrared image acquisition unit 20. According to an embodiment, the tactile image acquisition unit 10 can collect elasticity distribution information of a breast when a compression paddle squeezes the breast with a predetermined compression rate and the near-infrared image acquisition unit 20 can collect hemoglobin distribution information of a breast using the property of hemoglobin absorbing near-infrared wavelengths. The components are described in detail hereafter.

The tactile image acquisition unit 10 acquires tactile images by collecting elasticity distribution information of a breast and may include a tactile sensor 11. The tactile sensor 11 may be a pressure sensor or an optical tactile sensor, and depending on embodiments, it may be a piezoelectric sensor. The pressure sensor can acquire elasticity distribution of a breast using the difference in pressure between portions that are hardened or not when the breast is squeezed. The optical tactile sensor, a tactile sensor 400 based on optics, can acquire elasticity distribution of a breast. The optical tactile sensor may be composed of transparent silicon, a camera, and an LED. When the light source radiates light into the transparent silicon such that the light is totally reflected, the light has a scattered reflection when the silicon is deformed by the portions changed in elasticity. The camera can acquire an image from the light making a scattered reflection, and such an image is called a tactile image. It is possible to make a breast tissue elasticity map by applying a finite elements method, an inversion algorithm, and a forward algorithm to the elasticity distribution information acquired by the tactile image acquisition unit, using an artificial neural network.

The near-infrared image acquisition unit 20 can acquire a near-infrared image by radiating infrared light, to a breast using a lamp 21. Tumors consume more oxygen and have more blood vessels than common surrounding tissues. Accordingly, the amount of hemoglobin, which is protein in blood carrying oxygen, is different between a normal tissue and a tumor and it may be possible to determine whether there is a breast cancer from the level of hemoglobin based on this fact. According to an embodiment, it is possible to obtain a functional image showing a relative change in concentration of the entire blood and deoxidized hemoglobin (deoxyHb) using near-infrared light with a wavelength of 750 nm and 830 nm. The lamp 21 may be an LED, and near-infrared wavelengths from the LED permeate a breast tissue, whereby the quantity of hemoglobin in the breast tissue can be measured.

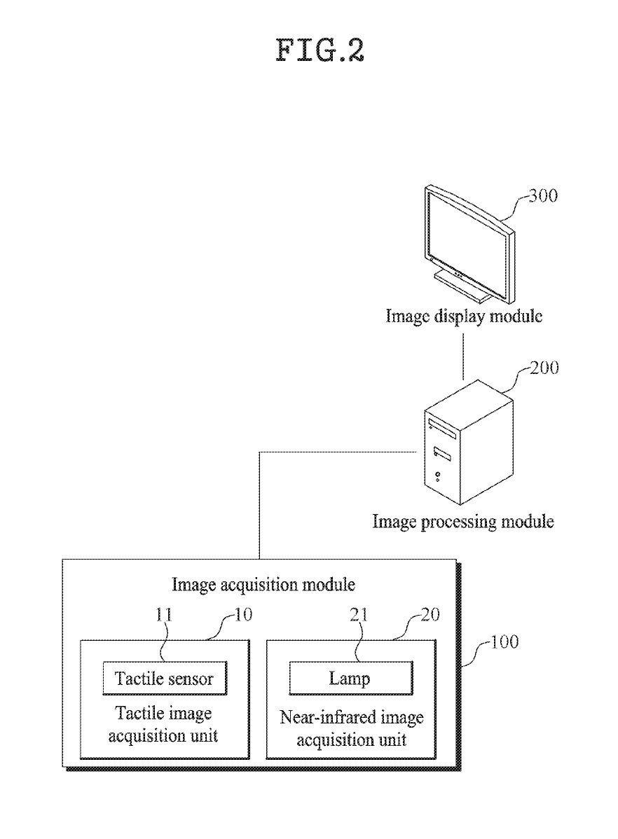

FIG. 2 is a diagram showing the configuration of an image diagnosis device for photographing a breast by using matching of a tactile image and a near-infrared image according to an embodiment of the present invention. As shown in FIG, 2, an image diagnosis device for photographing a breast by using matching of a tactile image and a near-infrared image according to an embodiment of the present invention may include an image acquisition module 100, an image processing module 200, and an image display module 300.

The image acquisition module 100 may include the tactile image acquisition unit 100 that acquires a tactile image by collecting elasticity distribution information of a breast and the near-infrared image acquisition unit 200 that acquires a near-infrared image by radiating near-infrared light to a breast. The tactile image acquisition unit 10 may include any one selected from a group including a pressure sensor, a piezoelectric sensor, and an optical tactile sensor and can collect elasticity distribution information of a squeezed breast when the breast is squeezed with a predetermined pressing rate by a compression paddle. The near-infrared image acquisition unit 20 can collect hemoglobin distribution information using the property of hemoglobin absorbing near-infrared wavelengths.

The image processing module 200 can match and show images acquired by the tactile image acquisition unit 10 and the near- infrared image acquisition unit 20 on the same coordinates. Preferably, it may be possible to match two images by mapping a tactile image and a near infrared image on a pre-stored breast model, in which the pre-stored breast model may be derived from an electronic medical record of a corresponding patient. That is, it is possible to show two images on the same coordinates by mapping a tactile image and a near-infrared image on a pre-stored breast model of a patient and then matching the images.

On the other hand, according to an embodiment of the present invention, the image processing module 200 may derive a color map in accordance with the intensity of elasticity using elasticity distribution information of a breast collected by the tactile image acquisition, unit 10 and may derive and match a hemoglobin map into one image using hemoglobin distribution information of a breast collected by the near-infrared image acquisition unit.

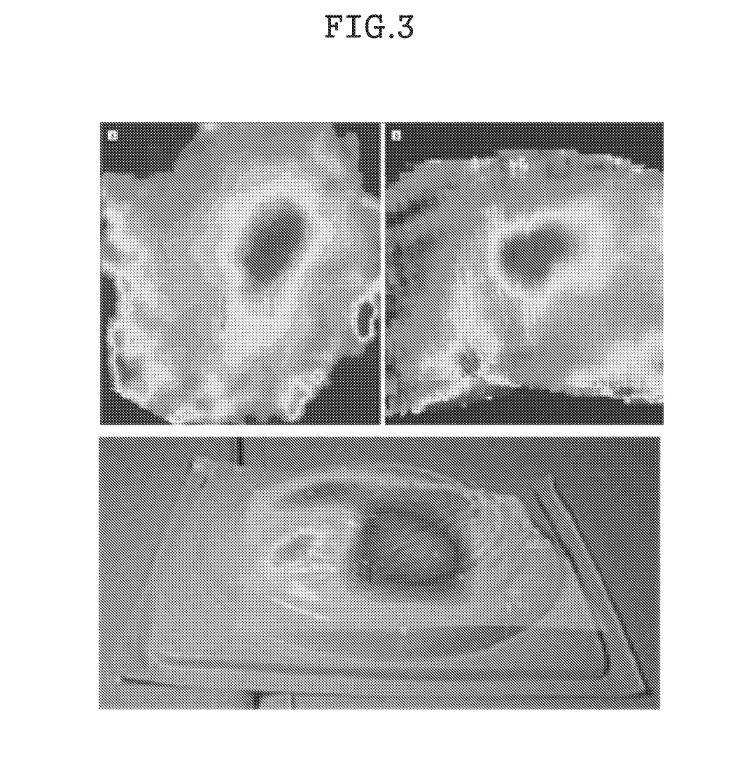

FIG. 3 is a picture of a tactile image acquired by an image diagnosis device for photographing a breast by using matching of a tactile image and a near-infrared image according to an embodiment of the present invention, an elasticity map created by mapping the tactile image to a breast model. As shown in FIG. 3, it is possible to create an elasticity map by mapping a tactile image acquired by an image diagnosis device for photographing a breast by using matching of a tactile image and a near-infrared image according to an embodiment of the present invention to a pre-stored breast model, and a portion where elasticity changes can be seen from the breast model. Further, qualitative elasticity can be measured and a color map can be produced in accordance with intensity of the elasticity.

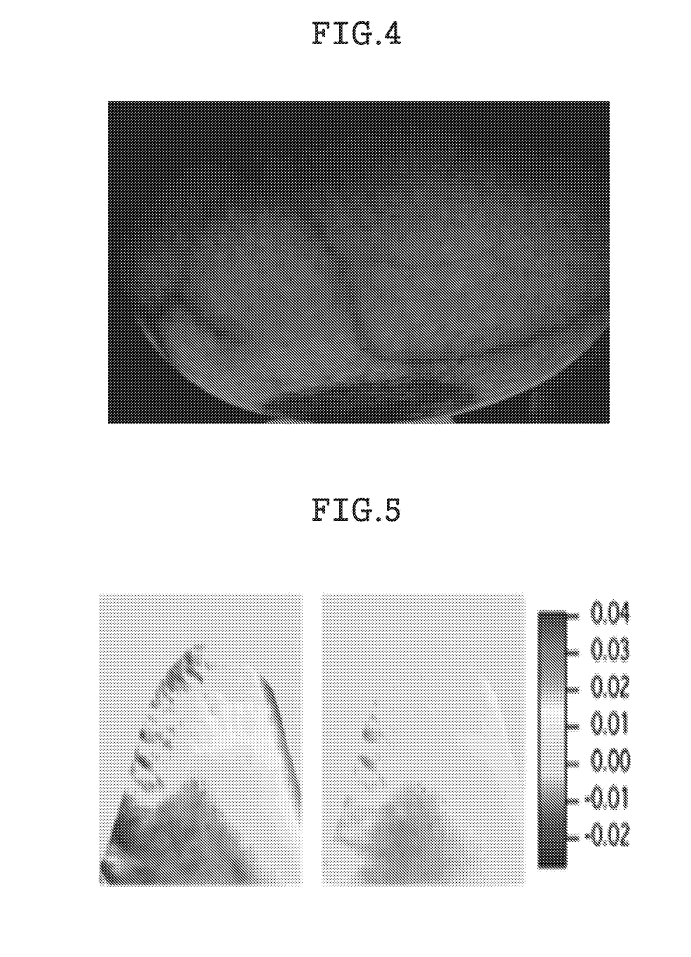

FIG. 4 is a picture of a near-infrared image acquired by an image diagnosis device for photographing a breast by using matching of a tactile image and a near-infrared image according to an embodiment of the present invention and FIG. 5 is a picture of a hemoglobin map crated from a near-infrared image acquired by an image diagnosis device for photographing a breast by using matching of a tactile image and a near-infrared image according to an embodiment of the present invention. FIG. 4 is a picture of an actual breast in the range of near-infrared light using transillumination, in which features of the internal tissue of the breast can be acquired because the skin of the breast can transmit near-infrared light at 2-3 mm. In particular, it is possible to create a hemoglobin map using the property of hemoglobin absorbing near-infrared light.

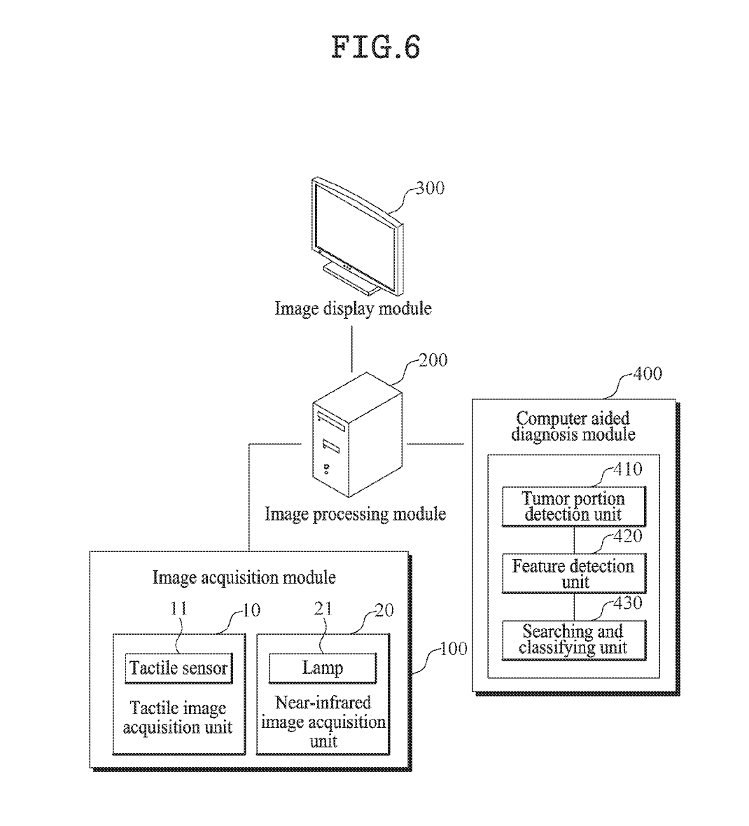

FIG. 6 is a diagram showing the configuration of an image diagnosis device for photographing a breast by using matching of a tactile image and a near-infrared image according to another embodiment of the present invention. As shown in FIG. 6, according to another embodiment of the present invention, an image diagnosis device for photographing a breast may further include a computer aided diagnosis module 400 including a tumor portion detection unit 410 that detects a portion where elasticity is a predetermined level or less or hemoglobin is a predetermined level or more from an image acquired by the image acquisition unit 100, a feature extraction unit 420 that extracts discriminated features by comparing the portion detected by the tumor detection unit 410 with a normal tissue, and a search & classifying unit 430 that searches and classifies features extracted by the feature extraction unit 420 through a search engine. It is possible to diagnose a breast cancer by automatically detecting a tumor portion, extracting the features of the tumor, and searching and classifying the tumor through the computer aided diagnosis module 400.

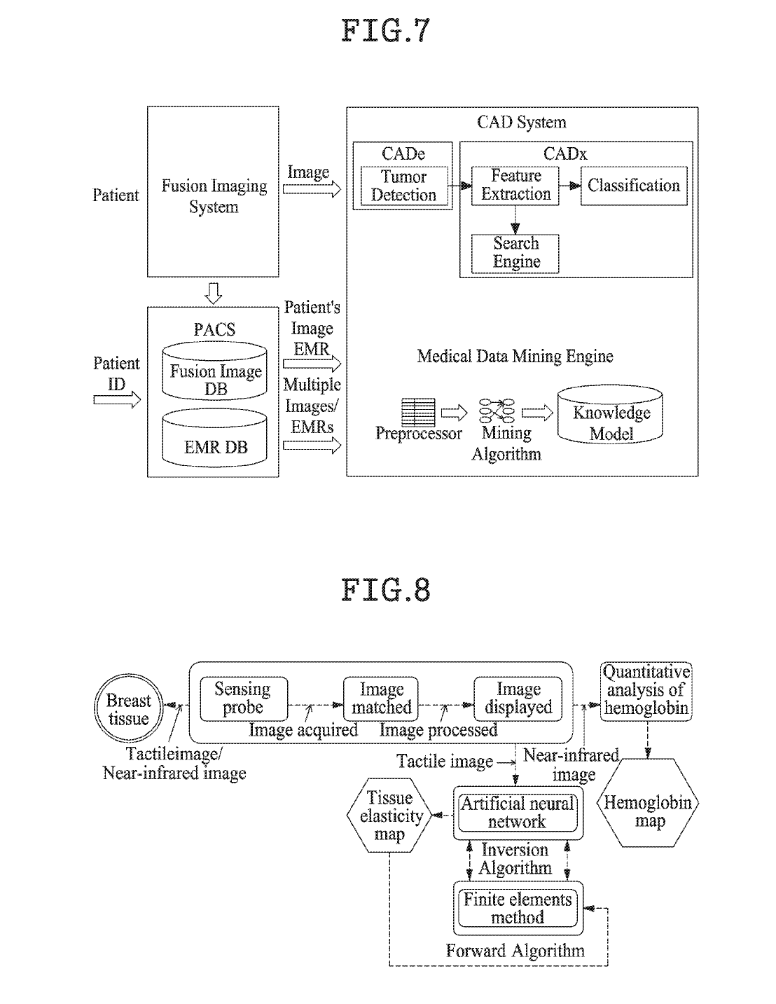

FIG. 7 is a diagram showing a process of image diagnosis for a breast by using matching of a tactile image and a near-infrared image according to another embodiment of the present invention. As shown in FIG. 7, a tactile image and a near-infrared image are matched from a patient, in which a breast model of a patient stored in an electronic medical record database can be used. It is possible to access the electronic medical record database using the patient's ID, but the present invention is not limited thereto. It is possible to examine elasticity and hemoglobin maps of a breast created by matching a tactile image and a near-infrared image using a computer aided diagnosis detection (CAD) system. The CAD system can examine whether there is a tumor and the width and depth of a tumor through processes of feature extraction for extracting features such as a shape and a property, which can be discriminated, searched, and classified.

That is, the present invention proposes an automated image diagnosis device that can create an en elasticity map and a hemoglobin map for the entire breast tissue from a composite image and can diagnose a breast cancer through an algorithm. It is possible to create an elasticity map of a breast and a near-infrared image showing the total amount of hemoglobin by calculating quantitative elasticity of a tissue and the width and depth of a tissue changed in elasticity, using a composite image. Further, it is possible to estimate and diagnose the period of breast cancer using a forward algorithm through finite element modeling on a tissue of a human body and an inversion algorithm based on machine-running that can teach the forward algorithm.

FIG. 8 is a diagram showing a diagnosis system using an image diagnosis device for photographing a breast by using matching of a tactile image and a near-infrared image according to another embodiment of the present invention. As shown in FIG. 8, a tactile image and a near-infrared image acquired by the sensing probe undergo image processing such as matching and are displayed on the image display module (display) 300. The near-infrared image is used to create a hemoglobin map through quantitative analysis on hemoglobin and the tactile image is used to create an elasticity map of a tissue. In detail, elasticity distribution information of a breast acquired by the tactile image acquisition unit can be made into a breast tissue elasticity map by applying a finite elements method, an inversion algorithm, and a forward algorithm to the elasticity distribution information acquired by the tactile image acquisition unit, using an artificial neural network. It is possible to check the state of the entire breast tissue using the hemoglobin map and the tissue elasticity map.

The image display module 300, which processes and displays images, may include any one selected from a group including a CRT, an LCD, an LED, an OLED, and a PDP.

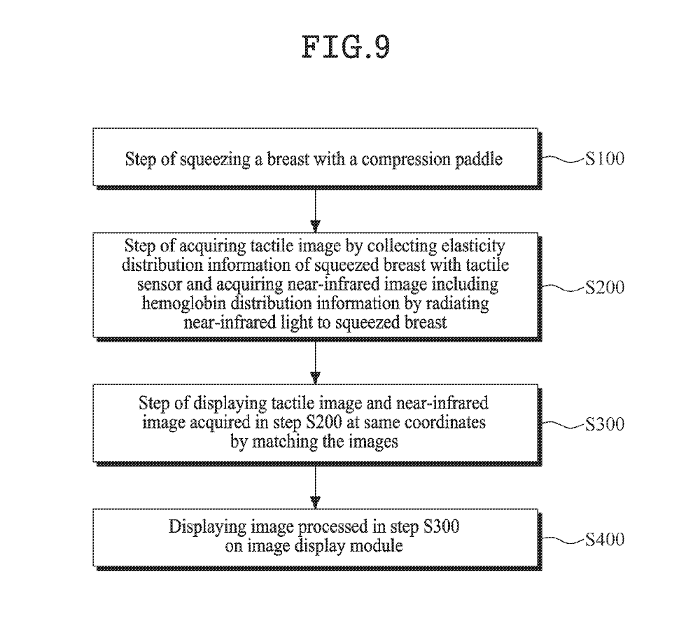

FIG. 9 is a flowchart showing a method for acquiring a breast tissue image by using matching of a tactile, image and a near-infrared image according to an embodiment of the present invention. As shown in FIG. 9, a method for acquiring a breast tissue image by using matching of a tactile image and a near-infrared image according to an embodiment of the present invention may include: a step of squeezing a breast with a compression paddle (S100); a step of acquiring a tactile image by collecting elasticity distribution information of the squeezed breast through a tactile sensor and acquiring a near-infrared image including hemoglobin distribution information of the breast (S200); a step of matching and displaying the tactile image and the near-infrared image acquired in the step S200 on the same coordinates (S300); and a step of displaying the images processed in the step S300 on an image display module (S400).

The step 2200 may further include a step of deriving a color map in accordance with intensity of elasticity using the elasticity distribution information of the breast and deriving a hemoglobin map using the hemoglobin distribution information of the breast. Further, depending on embodiments, it may further include a step of making the elasticity distribution information of the breast into a breast tissue elasticity map by applying a finite elements method, an inversion algorithm, and a forward algorithm to the elasticity distribution information acquired by the tactile image acquisition unit, using an artificial neural network.

The step S300 may be a step of matching two images by mapping a tactile image and a near-infrared image to a pre-stored breast model, which the pre stored breast model may be derived from an electronic medical record of the corresponding patient.

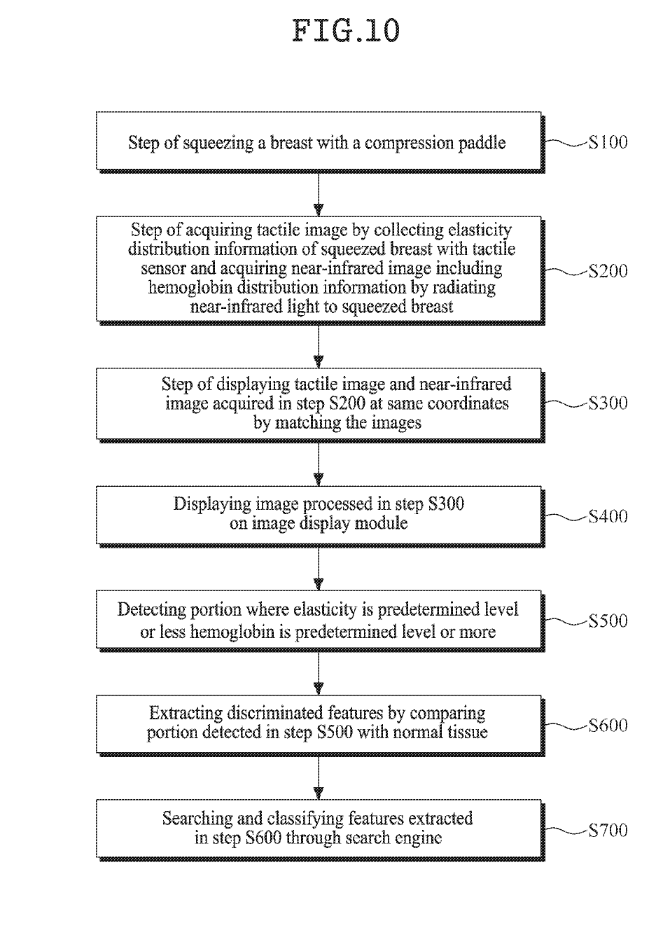

FIG. 10 is a method for acquiring a breast tissue image by using matching of a tactile image and a near-infrared image according to another embodiment of the present invention. As shown in FIG. 10, according to another embodiment of the present invention, the method may further include, after the step S400, a step of detecting a portion where elasticity is a predetermined level or less or hemoglobin is a predetermined level or more (S500), a step of extracting a discriminated feature between the portion detected in the step S500 and a normal tissue (S600), and a step (S700) of searching and classifying the feature extracted in the step S600 through a search engine.

The method for acquiring a breast tissue image by using matching of a tactile image and a near-infrared image proposed in the present invention may be used for animals. The steps in FIGS. 9 and 10 are similar to those described above with reference to FIGS. 1 to 8, so the detailed description is not provided.

The present invention described above may be changed and modified in various ways by those skilled in the art and the scope of the present invention should be determined up the following claims.

1-3. (canceled)

4. An image diagnosis device for photographing breast by using matching of a tactile image and a near-infrared image, the device comprising:
an image acquisition module including a tactile image acquisition unit acquiring a tactile image by collecting elasticity distribution information of a breast through a tactile sensor and a near-infrared image acquisition unit acquiring a near-infrared image by radiating near-infrared light to the breast;
an image processing module matching and displaying the images acquired by the tactile image acquisition unit and the near-infrared image acquisition unit on same coordinates; and
an image display module displaying the images processed by the image processing module.

5. The image diagnosis device of claim 4, wherein the tactile sensor of the tactile image acquisition unit is one selected from the group consisting of a pressure sensor, a piezoelectric sensor, and an optical tactile sensor.

6. The image diagnosis device of claim 4, wherein the tactile image acquisition unit collect elasticity distribution information of a squeezed breast when a compression paddle squeezes the breast with a predetermined compression rate, and the near-infrared image acquisition unit collects hemoglobin distribution information of a breast using a property of hemoglobin absorbing near-infrared wavelengths.

7. The image diagnosis device of claim 6, wherein the image processing module derives a color map in accordance with intensity of elasticity using the elasticity distribution information of a breast collected by the tactile image acquisition unit and derives a hemoglobin map using the hemoglobin distribution information of a breast collected by the near-infrared image acquisition unit.

8. The image diagnosis device of claim 4, wherein the image processing module matches two images by mapping the tactile image and the near-infrared image to pre-stored breast model.

9. The image diagnosis device of claim 8, wherein the pre-stored breast model is derived from an electronic medical record of a corresponding patient.

10. The image diagnosis device of claim 4, wherein the image processing module makes the elasticity distribution information of a breast acquired by the tactile image acquisition unit into a breast tissue elasticity map by applying a finite elements method, an inversion algorithm, and a forward algorithm to the elasticity distribution information, using an artificial neural network.

11. The image diagnosis device of claim 4, wherein the image display module includes one selected from the group consisting of a CRT, an LCD, an LED, an OLED, and a PDP.

12. The image diagnosis device of claim 4, further comprising a computer aided diagnosis module including: a tumor detection unit detecting a portion where elasticity is a predetermined level or less or hemoglobin is a predetermined level or more from an image acquired by the image acquisition unit; a feature extraction unit extracts discriminated features by comparing the portion detected by the tumor detection unit with a normal tissue; and a search and classifying unit searching and classifying features extracted by the feature extraction unit through a search engine.

13. A method of for acquiring a breast tissue image by using matching of a tactile image and a near-infrared image, the method comprising:
(1) a step of squeezing a breast with a compression paddle;
(2) a step of acquiring a tactile image by collecting elasticity distribution information of the squeezed breast through a tactile sensor and acquiring a near-infrared image including hemoglobin distribution information of the breast;
(3) a step of matching and displaying the tactile image and the near-infrared image acquired in the step (2) on same coordinates; and
(4) a step of displaying the images processed in the step (3) on an image display module.

14. The method of claim 13, wherein the tactile sensor in the step (2) is one selected from the group consisting of a pressure sensor, a piezoelectric sensor, and an optical tactile sensor.

15. The method of claim 13, wherein the step (2) includes a step of deriving a color map in accordance with intensity of elasticity using the elasticity distribution information of a breast and deriving a hemoglobin map using the hemoglobin distribution information of a breast.

16. The method of claim 13, wherein the step (2) includes a step of making the elasticity distribution information of a breast acquired by the tactile image acquisition unit into a breast tissue elasticity map by applying a finite elements method, an inversion algorithm, and a forward algorithm to the elasticity distribution information, using an artificial neural network.

17. The method of claim 13, wherein the step (3) is a step of matching two images by mapping the tactile image and the near-infrared image to pre-stored breast model.

18. The method of claim 17, wherein the pre-stored breast model is derived from an electronic medical record of a corresponding patient.

19. The method of claim 13, further comprising: after the step (4),
(5) detecting a portion where elasticity is a predetermined level or less or hemoglobin is a predetermined level or more;
(6) extracting discriminated features by comparing the portion detected in the step (5) with a normal tissue; and
(7) searching and classifying the features extracted in the step (6) through a search engine.

20. The method of claim 13, wherein the method is used for animals.
